(19) United States
(12) Patent Application Publication
(10) Pub. No.: US 2026/0027119 A1
(43) Pub. Date: Jan. 29, 2026

(54) INHIBITORS OF CALCIUM/CALMODULIN-DEPENDENT PROTEIN KINASE II AND THEIR USES

(71) Applicant: The Johns Hopkins University, Baltimore, MD (US)

(72) Inventors: Oscar Eduardo Reyes Gaido, Baltimore, MD (US); Mark Anderson, Baltimore, MD (US)

(21) Appl. No.: 18/996,718

(22) PCT Filed: Jul. 19, 2023
(86) PCT No.: PCT/US2023/028135

§ 371 Date: Jan. 17, 2025

Related U.S. Application Data
(60) Provisional application No. 63/390,789, filed on Jul. 20, 2022.

(51) Int. Cl. A61K 31/519; A61K 31/4709; A61K 31/4745; A61K 31/506

(52) U.S. Cl. CPC A61K 31/519; A61K 31/4709; A61K 31/4745; A61K 31/506

(57) ABSTRACT

Calcium/calmodulin-dependent protein kinase II (CaMKII) inhibitors and their use in treating or preventing cardiovascular diseases are disclosed.

### FEDERALLY SPONSORED RESEARCH OR DEVELOPMENT

This invention was made with government support under grant HL 140034 awarded by the National Institutes of Health. The government has certain rights in the invention.

### BACKGROUND

Cardiovascular diseases remain as leading killers with more than 500 million cases of cardiovascular disease and 18 million deaths every year. The Ca²⁺ and Calmodulin dependent protein kinase II (CaMKII) is one of the most validated and established drivers of cardiac disease and injury. A wealth of literature has demonstrated the harmful role of CaMKII hyperactivity in several cardiac diseases including, but not limited to, heart failure, cardiac hypertrophy, atrial fibrillation, myocardial infarction, inherited arrhythmias, and cardiomyopathy. Inhibition of CaMKII is cardioprotective in multiple animal models. Furthermore, people with heart failure show excessive CaMKII activity despite β-adrenergic blockade. Accordingly, CaMKII inhibitors have been highly sought after for decades. Despite this medical need, no drugs that target CaMKII are currently clinically-approved.

### SUMMARY

In some aspects, the presently disclosed subject matter provides a method for treating or preventing a cardiovascular disease in subject in need of treatment thereof, the method comprising administering to the subject a therapeutically effective amount of a calcium/calmodulin-dependent protein kinase II (CaMKII) inhibitor.

In certain aspects, the cardiovascular disease is selected from heart failure, cardiac hypertrophy, atrial fibrillation, myocardial infarction, an inherited arrhythmia, cardiomyopathy, ventricular arrhythmia, atherosclerosis, restenosis, and combinations thereof. In certain aspects, the cardiovascular disease is associated with cardiotoxicity arising from drug therapy, heart attack, ischemia-reperfusion injury, or catecholaminergic polymorphic ventricular tachycardia, and combinations thereof.

In certain aspects, the CaMKII inhibitor is selected from the group consisting of Ruxolitinib, Baricitinib, Crenolanib, Silmitasertib, and Abemaciclib.

In certain aspects, the CaMKII inhibitor is administered prophylactically. In particular aspects, the prophylactic administration of the CaMKII inhibitor is cardioprotective.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, FIG. 1C, and FIG. 1D show high-throughput screening to identify CaMKII inhibitors. A stable human cell line that expresses CaMKAR and constitutively active human CaMKIIδ (cardiac isoform) was developed. This cell line sensitively reports CaMKII inhibition, as demonstrated by treatment with tool compound AS100397 (FIG. 1A). In these cells, the JHDLv3 library, which contains 4,475 compounds that are approved for human use, was screened (FIG. 1B). This primary screen identified 118 candidate compounds, which were subscreened using a CaMKAR in vitro assay (FIG. 1C). Validation of these hits in HEK293 cells and by a third-party laboratory led to five potent inhibitors. Notably, both screening steps recovered the positive control: pan-kinase inhibitor Staurosporine. Most importantly, none of the hits are originally designed to target kinases in the CAMK superfamily (FIG. 1D); three of the hits (Baricitinib, Ruxolitinib, Crenolanib) are designed to target tyrosine kinases which are farthest away from CAMK by kinase domain sequence homology. This results speaks to the unexpected nature of these findings, and that rational design could not have predicted that these compounds also would be CaMKII inhibitors;

FIG. 2 is an independent confirmation of CaMKII inhibition. Eurofins ScanELECT service was contracted to screen each compound at 5 μM against purified CaMKIIδ;

FIG. 3A and FIG. 3B demonstrate that in HEK293 cells expressing constitutively active CaMKIIδ, three compounds were more potent that tool inhibitor AS100397 (FIG. 3A). Moreover, four compounds were less cytotoxic (FIG. 3B). These data indicate that Ruxolitinib and Baricitinib appear to be more potent and less toxic than the best tool inhibitor;

FIG. 4A, FIG. 4B, and FIG. 4C show compounds that inhibit CaMKII in cardiomyocytes. CaMKII is stimulated in cardiac cells by simulating tachycardia with field pacing. All five compounds inhibit CaMKII activation at a high dose (FIG. 4A). When drug concentration is adjusted to match their respective maximum human plasma concentration, four out of the five compounds were able to ameliorate CaMKII (FIG. 4B, FIG. 4C). This result suggests that Silmitasertib, Abemaciclib, Crenolanib, and Ruxolitinib could inhibit CaMKII therapeutically at their current human doses;

FIG. 5A and FIG. 5B show that Ruxolitinib is over 10-fold more potent than DiOHF, a CaMKII inhibitor that was recently tested in humans (FIG. 5A). Ruxolitinib appeared to be similarly safe to cardiomyocytes as DiOHF and much less toxic than other CaMKII inhibitors (FIG. 5B); and

FIG. 6 demonstrates that Ruxolitinib inhibits cardiac CaMKII in vivo. Further, pretreatment with Ruxolitinib prevents isoproterenol-induced CaMKII activity and subsequent phosphorylation of its target phospholamban (residue threonine 17) in C57BL/6j wild-type mice.

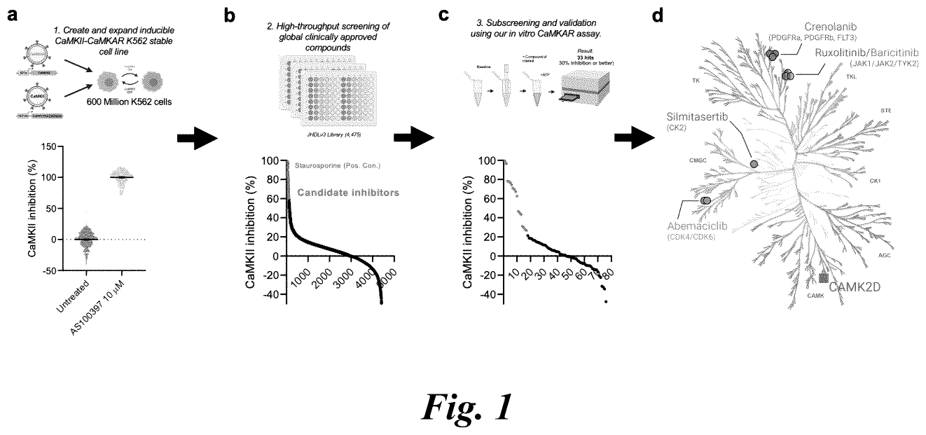

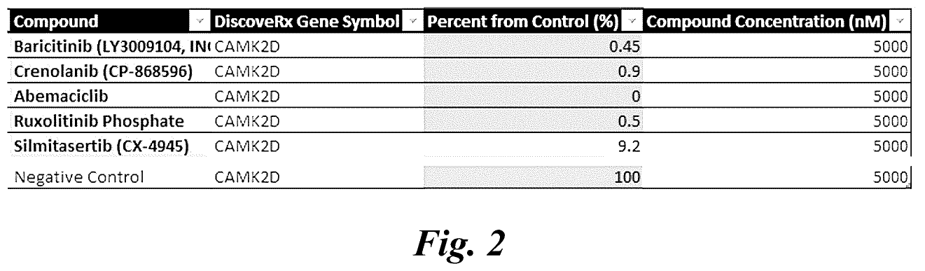

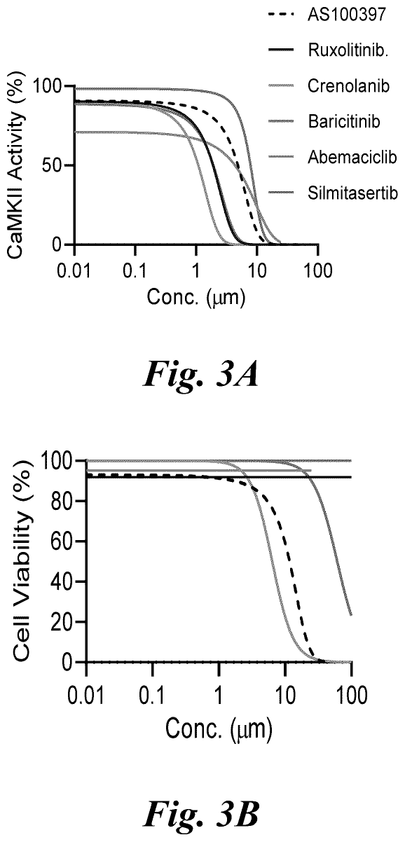

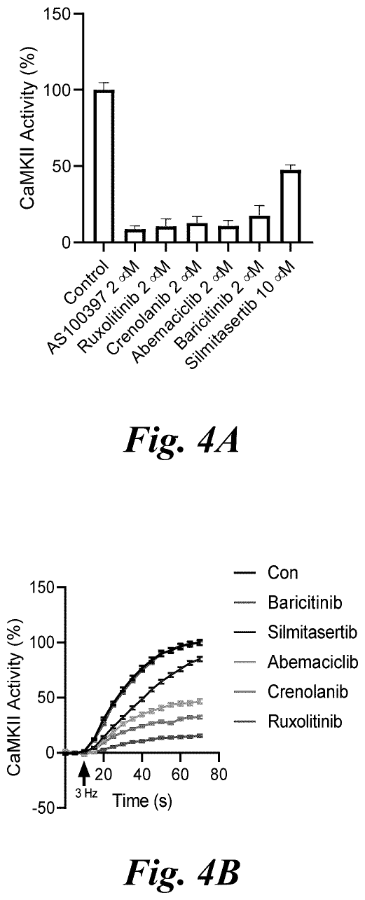

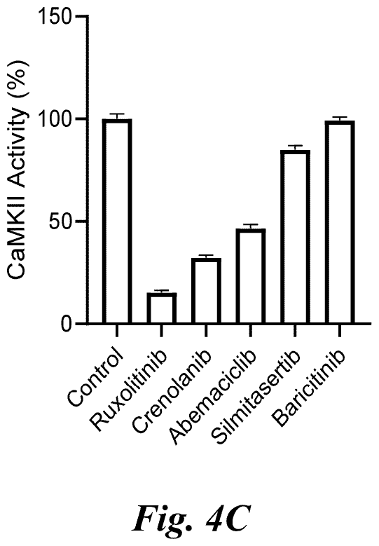

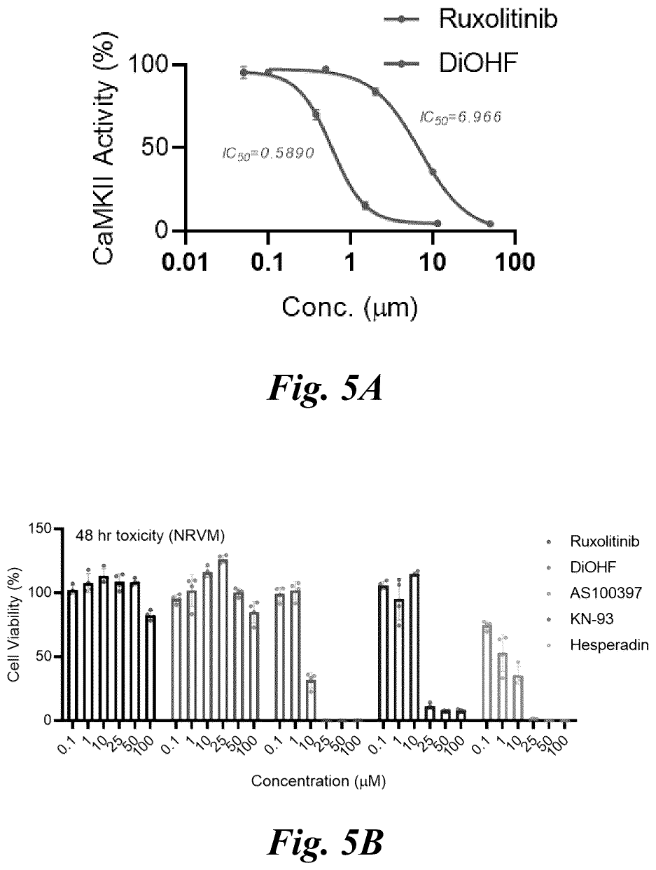

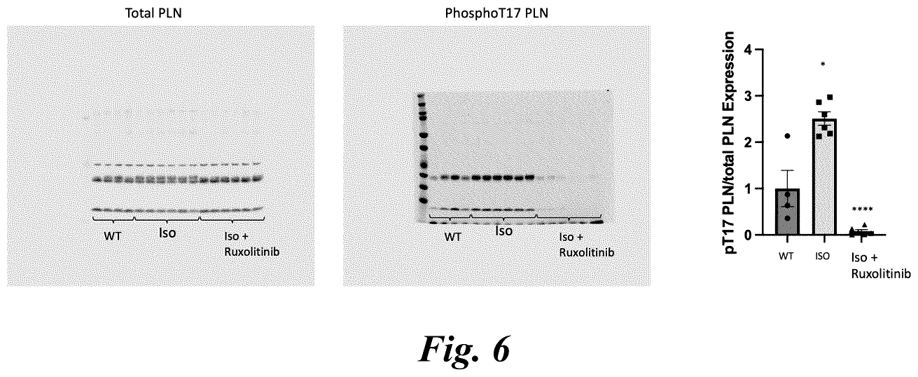

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides a method for treating or preventing a cardiovascular disease in subject in need of treatment thereof, the method comprising administering to the subject a therapeutically effective amount of a calcium/calmodulin-dependent protein kinase II (CaMKII) inhibitor.

In certain embodiments, the CaMKII inhibitor inhibits one or more isoforms of CaMKII, including one or more of the alpha, beta, delta, and gamma isoforms of CaMKII.

The CaMKII inhibitor that is administered in the method may inhibit CaMKII directly (e.g., by directly inhibiting the kinase activity of CaMKII) or indirectly (e.g., by inhibiting activation or expression of CaMKII).

As used herein, the term “inhibit” means to decrease or diminish the excess PSMA activity found in a subject. The term “inhibit” also may mean to decrease, suppress, attenuate, diminish, arrest, or stabilize the development or progression of a disease or condition. Inhibition may occur, for e.g., by at least 10%, 20%, 30%, 40%, 50%, 60%, 70%, 80%, 90%, 95%, 98%, 99%, or even 100% compared to an untreated control subject or a subject without the disease or disorder.

As used herein, “CaMKII” refers to the enzyme “calcium/calmodulin dependent protein kinase II.” In humans, there are four separate, highly homologous genes for CaMKII called alpha, beta, delta, or gamma. Multiple isoforms of these genes are expressed through alternative splicing mechanisms. Representative sequences for the isoforms of these genes have been submitted to public depositories such as GenBank and include: GenBank Accession No. NP_741960. CaMKII alpha isoform 2; GenBank Accession No. NP_057065, CaMKII alpha isoform 1; GenBank Accession No. NP_742079, CaMKII beta isoform 6: GenBank Accession No. NP_742080, CaMKII beta isoform 7: GenBank Accession No. NP742077. CaMKII beta isoform 4: GenBank Accession No. NP001211, CaMKII beta isoform 1; GenBank Accession No. NP_742081. CaMKII beta isoform 8: GenBank Accession No. NP_742078. CaMKII beta isoform 5: GenBank Accession No. NP_742076. CaMKII beta isoform 3: GenBank Accession No. NP_742075. CaMKII beta isoform 2; GenBank Accession No. NP_001212, CaMKII delta isoform 3: GenBank Accession No. NP_742126, CaMKII delta isoform 2: GenBank Accession No. NP_742125. CaMKII isoform 1; GenBank Accession No. NP_742113, CaMKII isoform 1; GenBank Accession No. NP_001020609, CaMKII delta isoform 2 (SEQ ID NO:12); NP_751910, CaMKII gamma isoform 3; GenBank Accession No. NP751913, CaMKII gamma isoform 6: GenBank Accession No. NP_751913. CaMKII gamma isoform 6; GenBank Accession No. NP_751911, CaMKII gamma isoform 1; GenBank Accession No. NP_751909, CaMKII gamma isoform 2; GenBank Accession No. NP_751909, CaMKII gamma isoform 2; GenBank Accession No. NP_001213, CaMKII gamma isoform 4; all of which GenBank entries are incorporated herein by reference in their entireties.

In certain embodiments, the cardiovascular disease is selected from heart failure, cardiac hypertrophy, atrial fibrillation, myocardial infarction, an inherited arrhythmia, cardiomyopathy, ventricular arrhythmia, atherosclerosis, restenosis, and combinations thereof. In certain embodiments, the cardiovascular disease is associated with cardiotoxicity arising from drug therapy, heart attack, ischemia-reperfusion injury, or catecholaminergic polymorphic ventricular tachycardia, and combinations thereof.

In certain embodiments, the CaMKII inhibitor is selected from the group consisting of:

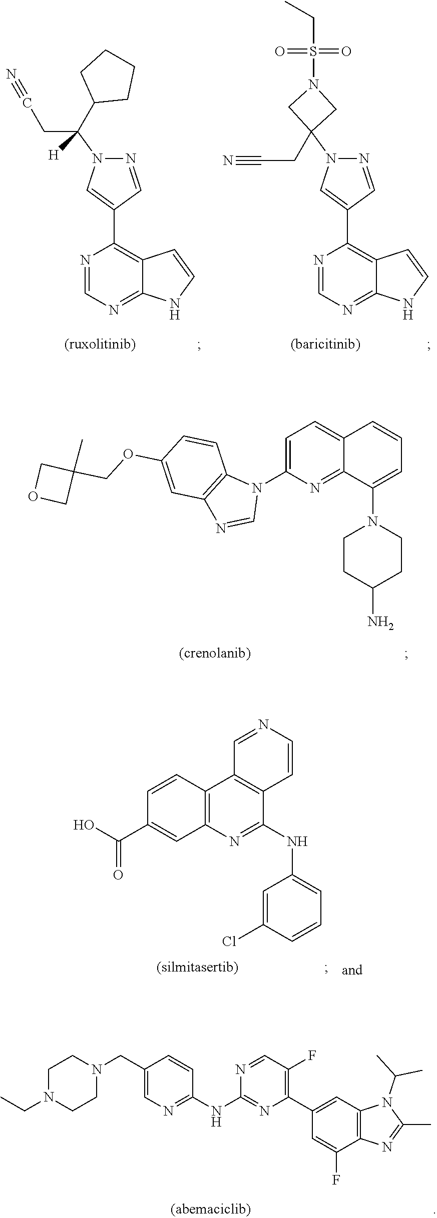 [SMILES: N#CC[C@H](C1CCCC1)n1cc(-c2ncnc3[nH]ccc23)cn1 ; CCS(=O)(=O)N1CC(CC#N)(n2cc(-c3ncnc4[nH]ccc34)cn2)C1 ; CC1(COc2ccc3c(c2)ncn3-c2ccc3cccc(N4CCC(N)CC4)c3n2)COC1 ; O=C(O)c1ccc2c(c1)nc(Nc1cccc(Cl)c1)c1ccncc12 ; CCN1CCN(Cc2ccc(Nc3ncc(F)c(-c4cc(F)c5nc(C)n(C(C)C)c5c4)n3)nc2)CC1]

In certain embodiments, the CaMKII inhibitor is administered prophylactically. In particular embodiments, the prophylactic administration of the CaMKII inhibitor is cardioprotective.

As used herein, the term “treating” can include reversing, alleviating, inhibiting the progression of, preventing, or reducing the likelihood of the disease, disorder, or condition to which such term applies, or one or more symptoms or manifestations of such disease, disorder, or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or refers to the amount necessary to elicit the desired biological response. As will be appreciated by those of ordinary skill in this art, the effective amount of an agent may vary depending on such factors as the desired biological endpoint, the agent to be delivered, the makeup of the pharmaceutical composition, the drug target, and the like.

The term “combination” is used in its broadest sense and means that a subject is administered one agent, more particularly an CaMKII inhibitor in combination with one or more therapeutic agents. More particularly, the term “in combination” refers to the concomitant administration of a CaMKII inhibitor and one or more therapeutic agents for the treatment of a single disease state. As used herein, the CaMKII inhibitor and one or more additional therapeutic agents may be combined and administered at the same time, or may be administered alternately or sequentially on the same or separate days.

Further, the presently disclosed CaMKII inhibitors in combination with an additional one or more therapeutic agents can be further administered with adjuvants that enhance stability of the agents, alone or in combination with the agent, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of the presently disclosed CaMKII inhibitors in combination with the one or more additional therapeutic agents can be varied so long as the beneficial effects of the combination of the agent and additional one or more therapeutic agents are achieved. Accordingly, the phrase “in combination with” refers to the administration of an CaMKII inhibitor described herein and one or more additional therapeutic agents either simultaneously, sequentially, or a combination thereof. Therefore, a subject administered a combination of a presently disclosed CaMKII inhibitor and an additional one or more therapeutic agents can receive an CaMKII inhibitor and additional one or more therapeutic agents at the same time (i.e., simultaneously) or at different times (i.e., sequentially, in either order, on the same day or on different days), so long as the effect of the combination of the agent and additional one or more therapeutic agents is achieved in the subject.

When administered sequentially, the CaMKII inhibitor and additional one or more therapeutic agents can be administered within 1, 5, 10, 30, 60, 120, 180, 240 minutes or longer of one another. In other embodiments, the CaMKII inhibitor and additional one or more therapeutic agents can be administered sequentially, can be administered within 1, 5, 10, 15, 20 or more days of one another.

When administered in combination, the effective dosage of the agent to elicit a particular biological response may be less than the effective dosage of the agent when administered alone, thereby allowing a reduction in the dose of the agent relative to the dose that would be needed if the agent was administered as a single agent. The effects of the agent along with the additional one or more therapeutic agents, but need not be, additive or synergistic. The agent and/or the additional one or more therapeutic agents may be administered multiple times.

In some embodiments, when administered in combination, the agent and the additional one or more therapeutic agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( SI )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, +100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Inhibitors Of Calcium/Calmodulin-Dependent Protein Kinase II

In some embodiments, the presently disclosed subject matter provides a CaMKII activity biosensor, referred to herein as a CaMKII Activity Reporter (CaMKAR), that is tractable for high-throughput screens. Using this sensor, a library of compounds that are safe for human use (i.e., Food and Drug Administration (FDA)-, European Medicines Agency (EMA)-, China Food and Drug Administration (CFDA)-, and Pharmaceuticals and Medical Devices Agency (PMDA)-approved compounds) was screened for previously unidentified CaMKII inhibitors. Five compounds that potently inhibited cardiac CaMKII (CaMKIIδ) in human cells were identified: Ruxolitinib, Baricitinib, Crenolanib, Silmitasertib, and Abemaciclib. In rat cardiac cells, all five compounds prevented CaMKII activity. Notably, none of these compounds are currently used to treat cardiac illnesses or are intended to target CaMKII.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
U.S. Pat. No. 9,951,061 for CaMKII inhibitors and uses thereof, to Levy et al., issued Apr. 24, 2018.
U.S. Pat. No. 10,759,792 for CaMKII inhibitors and uses thereof, to Levy et al., issued Sep. 1, 2020.
U.S. Pat. No. 11,325,908 for CaMKII inhibitors and uses thereof, to Levy et al., issued May 10, 2022.
U.S. Pat. No. 8,975,250 for 5 oxo-5,8-dihydropyrido[2,3-d]pyrimidine derivatives as CaMKII kinase inhibitors for treating cardiovascular diseases, to Beauverger, et al., issued Mar. 10, 2015.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for treating or preventing a cardiovascular disease in subject in need of treatment thereof, the method comprising administering to the subject a therapeutically effective amount of a calcium/calmodulin-dependent protein kinase II (CaMKII) inhibitor.

2. The method of claim 1, wherein the cardiovascular disease is selected from heart failure, cardiac hypertrophy, atrial fibrillation, myocardial infarction, an inherited arrhythmia, cardiomyopathy, ventricular arrhythmia, atherosclerosis, restenosis, and combinations thereof.

3. The method of claim 1, wherein the cardiovascular disease is associated with cardiotoxicity arising from drug therapy, heart attack, ischemia-reperfusion injury, or catecholaminergic polymorphic ventricular tachycardia, and combinations thereof.

4. The method of claim 1, wherein the CaMKII inhibitor is selected from the group consisting of Ruxolitinib, Baricitinib, Crenolanib, Silmitasertib, and Abemaciclib.

5. The method of claim 1, wherein the CaMKII inhibitor is administered prophylactically.

6. The method of claim 5, wherein the prophylactic administration of the CaMKII inhibitor is cardioprotective.
